(19) United States
(12) Patent Application Publication
(10) Pub. No.: US 2022/0346726 A1
(43) Pub. Date: Nov. 3, 2022

(54) SHIFT COMPLIANCE MANAGEMENT SYSTEM FOR PREVENTING PRESSURE ULCERS

(71) Applicant: HiPR Innovation, Inc., Laredo, TX (US)

(72) Inventors: Harikrishan SACHDEV, Laredo, TX (US); Koushalya SACHDEV, Laredo, TX (US); David CHUN, Austin, TX (US); Samuel ZHANG, Richardson, TX (US)

(21) Appl. No.: 17/731,475

(22) Filed: Apr. 28, 2022

Related U.S. Application Data
(60) Provisional application No. 63/181,737, filed on Apr. 29, 2021.

(51) Int. Cl. A61B 5/00

(52) U.S. Cl. CPC A61B 5/743; A61B 5/0022; A61B 5/0024; A61B 5/6894; A61B 5/6843

(57) ABSTRACT

A shift compliance management system manages user compliance with recommendations for shift weight exercises to mitigate risk of a wheelchair user developing pressure ulcers. A sensing device includes an array of pressure sensors to sense the user's weight distribution and to detect timing and duration of weight shifts performed by the user. The sensing device communicates with a user application that assesses a compliance level of the sensed weight shifts with a set of weight shift recommendations for the patient. A compliance server collects patient data and may perform various analytics related to compliance. The analytical data may be presented to the wheelchair user and/or a caretaker to indicate compliance adherence.

### CROSS-REFERENCED TO RELATED APPLICATIONS

This application claims the benefit of U.S. Provisional Application No. 63/181,737 filed on Apr. 29, 2021, which is incorporated by reference herein.

### BACKGROUND

### Technical Field

The disclosed embodiments relate to a shift compliance management system for preventing pressure ulcers in wheelchair users.

### Description of Related Art

Wheelchair users are commonly at high risk of developing pressure ulcers caused by external pressure applied to a body part over a long period of time. Pressure ulcers can be painful, difficult to heal, and prone to infection. Wheelchair users can mitigate their risk of developing pressure ulcers by regularly conducting weight shift exercises to periodically relieve pressure from vulnerable areas. Nevertheless, many wheelchair users find it difficult to maintain an appropriate weight shifting routine.

### BRIEF DESCRIPTION OF THE DRAWINGS

FIG. 1A is a system environment for a shift compliance management.

FIG. 1B is a block diagram illustrating an example architecture of a shift compliance management system.

FIG. 2A is a block diagram illustrating an example embodiment of an architecture for a sensing device.

FIG. 2B is an example embodiment of a physical structure of a sensing device.

FIG. 3 is a block diagram illustrating an example embodiment of a processing device.

FIG. 4 is a block diagram illustrating an example embodiment of a compliance management application.

FIG. 5 is a flowchart illustrating an example embodiment of a process for facilitating shift compliance management.

FIG. 6 is a block diagram illustrating an example embodiment of a server application.

FIG. 7 is a messaging diagram illustrating an example connection protocol between a sensing device and a user device.

FIG. 8 is a flowchart illustrating an example embodiment of a process performed by a sensing device for obtaining pressure sensor data.

FIG. 9 is a flowchart illustrating an example embodiment of a process performed by a sensing device for communicating sensor data with a user device.

FIG. 10 is a flowchart illustrating an example embodiment of a process for managing collection and transmission of sensor data.

### DETAILED DESCRIPTION

The Figures (FIGS.) and the following description describe certain embodiments by way of illustration only. One skilled in the art will readily recognize from the following description that alternative embodiments of the structures and methods illustrated herein may be employed without departing from the principles described herein. Reference will now be made to several embodiments, examples of which are illustrated in the accompanying figures. Wherever practicable, similar or like reference numbers may be used in the figures and may indicate similar or like functionality.

A shift compliance management system manages user compliance with recommendations for shift weight exercises to mitigate risk of a wheelchair user developing pressure ulcers. A sensing device includes an array of pressure sensors to sense the user's weight distribution and to detect timing and duration of weight shifts performed by the user. The sensing device communicates with a compliance management application that assesses a compliance level of the detected weight shifts with a set of weight shift recommendations for the patient. A compliance server collects patient data and may perform various analytics related to compliance. The analytical data may be presented to the wheelchair user and/or a caretaker to indicate compliance adherence.

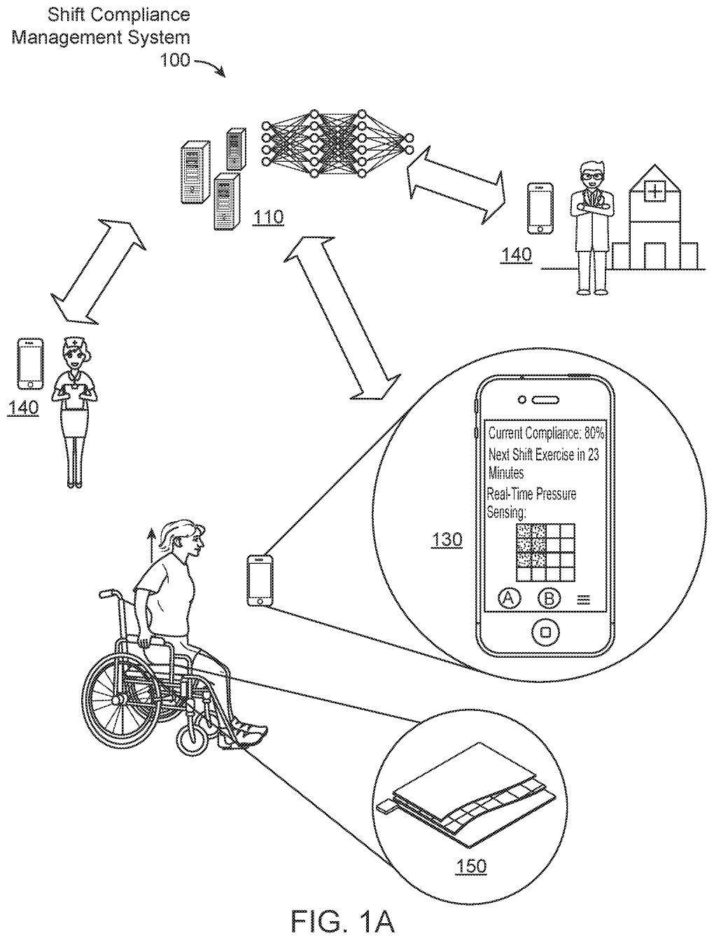

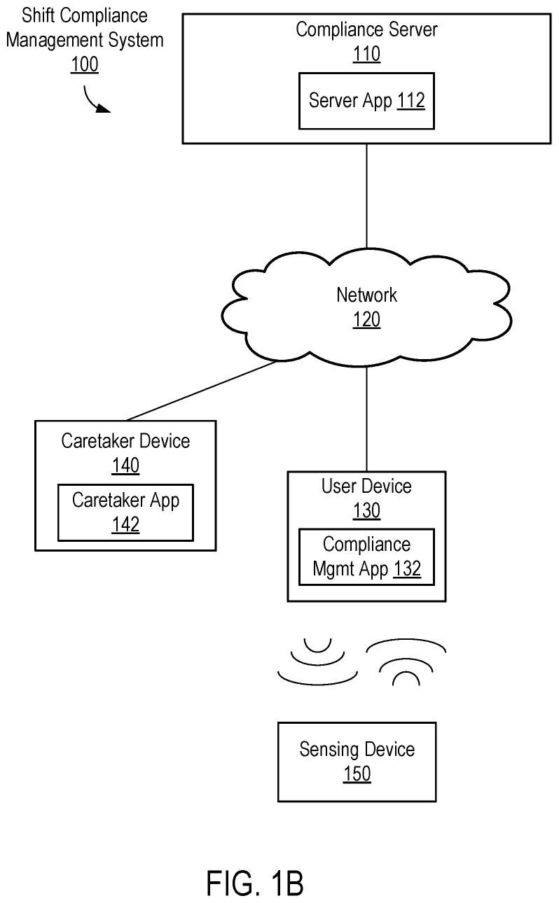

FIGS. 1A-B illustrate an example embodiment of a shift compliance management system 100. Particularly, FIG. 1A is illustrative of an overall user experience associated with the shift compliance management system 100 while FIG. 1B is a block diagram illustrating an example architecture. The shift compliance management system 100 comprises at least a sensing device 150, a user device 130 executing a compliance management application 132, one or more caretaker devices 140 executing a caretaker application 142, and compliance server 110 executing a server application 112. The user device 130, caretaker device 140, and compliance server 110 may be communicatively coupled via a network 120 which may include one or more local area networks (LANs), wide area networks (WANs), or direct wired or wireless connections. The user device 130 may furthermore be communicatively coupled with the sensing device 150 via a wired or wireless communication interface such as Bluetooth, Bluetooth Low Energy (BLE), WiFi Direct, or a local area network (LAN). In an embodiment, the sensing device 150 may instead be directly coupled to the network 120 and may communicate directly with the compliance server 110, caretaker device 140, and/or other network devices.

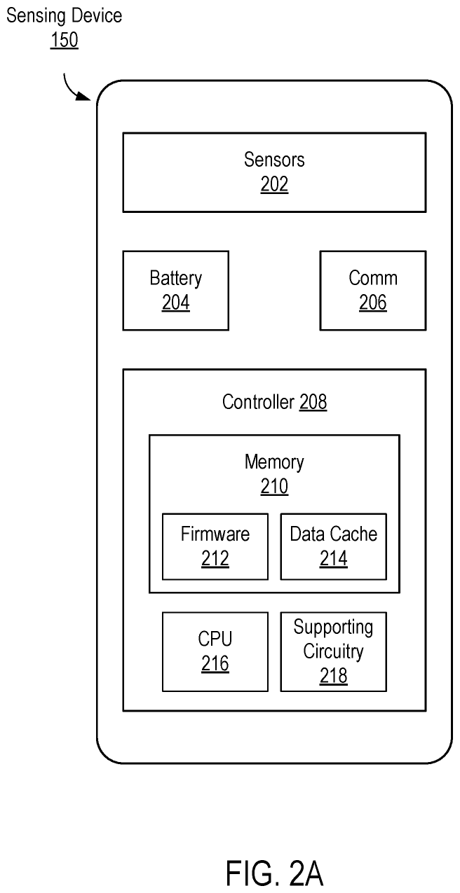

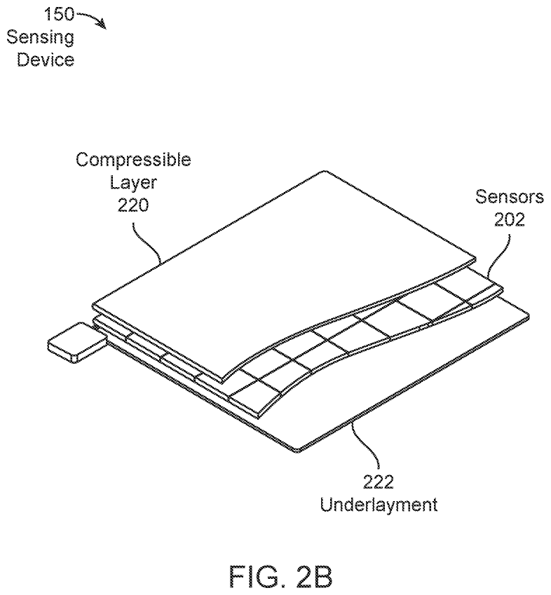

The sensing device 150 comprises a wheelchair-compatible pad, cushion, or other surface placed underneath the gluteal region of a wheelchair user. In different embodiments, the sensing device 150 may be placed either underneath or over a wheelchair seating pad. Furthermore, in various embodiments, the sensing device 150 may comprise a standalone device or may be integrated with a wheelchair seat or seating pad. The sensing device 150 includes a matrix of sensors for detecting pressure at different physical locations of the seat. The sensing device 150 may communicate the sensor data with the user device 130 to provide information relevant to detecting compliance with weight shift recommendations. An example embodiment of a sensing device 150 is described in further detail below with respect to FIGS. 2A-B.

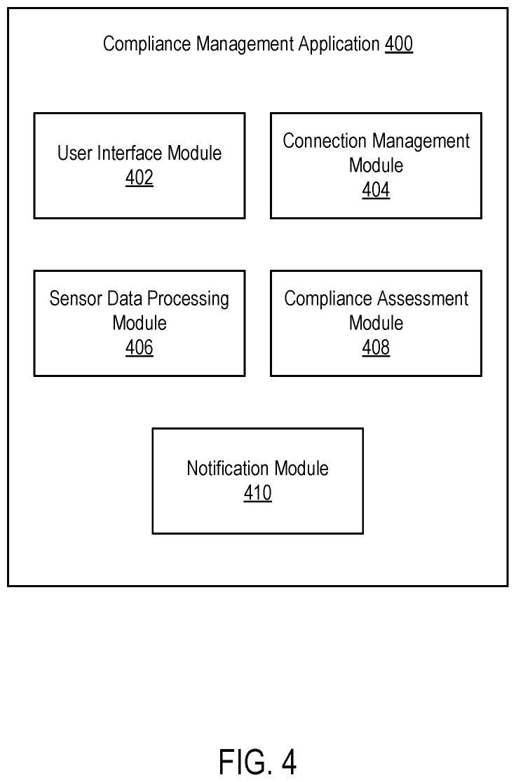

The user device 130 comprises a computing device such as a mobile phone, tablet, laptop computer, desktop computer, or other computing device capable of executing the compliance management application 132 and communicating with the sensing device 150 and the network 120. The compliance management application 132 generally obtains the sensor data from the sensing device 150, processes the sensor data to determine compliance with a set of weight shift recommendations, and provides notifications indicative of the user's compliance levels. In various embodiments, the compliance management application 132 may furthermore collect various biometric and/or demographic information about the user to customize the weight shift recommendations and/or notifications. Furthermore, the compliance management application 132 may communicate with the compliance server 110 to provide the collected data to the server 110 and to obtain various analytical data associated with user compliance. The compliance management application 132 may furthermore receive the compliance recommendations from the server 110. An example embodiment of a compliance management application 132 is described in further detail below with respect to FIG. 4.

The caretaker device 140 may comprise a mobile phone, tablet, laptop computer, desktop computer, or other computing device capable of executing the caretaker application 142 and communicating with the network 120. The caretaker application 142 may provide a user interface for a caretaker of a wheelchair user such as a physician, nurse, therapist, family member, or other individual granted access to the wheelchair user's compliance data. In some embodiments, the caretaker application 142 may provide similar notifications and/or analytical data as the compliance management application 132 (e.g., provided by the compliance server 110) associated with one or more users assigned to the caretaker application 142. Alternatively, the caretaker device 140 may gain access to a more limited set of data based on permissions granted via the compliance management application 132. The caretaker application 142 may furthermore enable the compliance server 110 to obtain various information from the caretaker such as, for example, specific shift recommendations for the patient, patient notes, notifications to be sent to the patient, or other information.

The compliance server 110 comprises a computing device executing a server application 112 for performing various data processing functions associated with shift compliance management as described herein. In various embodiments, the compliance server 110 may comprise one or more physical servers or one or more virtual servers. The compliance server 110 may be implemented as a standalone server or a set of distributed servers (e.g., a cloud computing environment). The compliance server 110 interfaces with one or more user applications 130 and/or caretaker applications 142 to facilitate various functions described herein. The compliance server 110 may maintain user accounts storing profile information for various users, provide remote storage for sensor data and/or other user data obtained from the compliance management application 132, and/or perform various computing functions to generate advanced analytical data based on the data histories for individual users or aggregated data histories from a population of users.

In an embodiment, the compliance server 110 may implement machine learning algorithms to learn relationships between the frequency and/or types of notifications presented by the compliance management application 132 and the observed user compliance levels responsive to the notifications. In another embodiment, the compliance server 110 learns which regions are subject to the highest pressure levels in various populations and may tailor the shift recommendations accordingly. The learning system may then update the frequency and/or types of notifications in a manner predicted to improve compliance. In embodiments where the compliance server 110 obtains user or caretaker feedback regarding occurrences of pressure ulcers, the compliance server 110 may learn relationships between the recommended or observed weight shift exercise schedule and the occurrences of pressure ulcers develop improved recommendations that may be based on patient-specific observations or observations across a population.

In alternative embodiments, functions attributed to the user device 130 may instead be performed remotely on the compliance server 110 or vice versa. In an embodiment, the compliance server 110 may be omitted and all functions may be performed on the user device 130. Alternatively, the user device 130 may be omitted and the sensing device 150 may communicate directly with the compliance server 110, which performs the functions of the compliance management application 132 described herein. In yet further embodiments, at least some functions of the compliance management application 132 or the compliance server 110 may instead be performed directly on the sensing device 150.

FIG. 2A is a block diagram of an architecture for an example sensing device 150 and FIG. 2B illustrates an example physical structure of the example sensing device 150. The sensing device 150 comprises a matrix of sensors 202, a battery 204, a communication interface 206, and a controller 208. The controller 208 may include one or more CPUs 216, a memory 210 (including firmware 212 and a data cache 214), and other supporting circuitry 218. The supporting circuitry 218 may include, for example, a Real Time Clock (RTC) or other time tracking mechanism, one or more input/output devices to facilitate real-time interactions such as a display, touch panel, buttons, or other input/output devices.

The sensors 202 include a matrix of pressure sensors for measuring real-time pressure or applied force. Examples of pressure sensors 202 may include Force Sensitive Resistors (FSR) pressure sensitive conductive sheets, or transduced-based pressure sensors. The sensors 202 may optionally include other types of sensors (either individual sensors or a matrix of sensors) for sensing other conditions such as, for example, weight, temperature, movement, moisture, or other biometric factors relating to the wheelchair user or the environment.

The battery 204 may comprise either a rechargeable or replaceable battery. Alternatively, the battery 204 may be omitted and the sensing device 150 may be powered from an external source.

The communication interface 206 comprises one or more wired and/or wireless interfaces for interfacing with the user device 130, the network 120, or another external device. The communication interface 206 may include, for example, a Bluetooth interface, a WiFi interface, a WiFi direct interface, a USB interface, or any other wired or wireless communication interfaces.

The controller 208 and CPU 216 may control various functions of the sensing device 150. For example, the controller 208 may store instructions that control timing of sensor readings from the sensors 202 performed by the CPU 216. In an embodiment, the CPU 216 may perform filtering or aggregating of sensor readings before sending them to the user device 130. For example, for each sensor 202, the CPU 216 may obtain a set of readings over a short time interval and average them before sending the average the reading to the user device 130. The CPU 216 may further control processes such as sensor calibration and/or initiation. The CPU 216 may also implement various power management controls such as switching the sensing device 150 between sleep and wake modes and may control timing of data transfers between the sensing device 150 and the user device 130 dependent on the mode. For example, the CPU 216 may cause sensor data to be read more frequently in the wake mode than in the sleep mode. The CPU 216 may further control association of various metadata with the sensor readings as will be further described below.

In the physical implementation of FIG. 2B, the sensing device 202 comprises a tiered cushion having multiple layers including a protective compressible layer 220 on the upper surface (e.g., a foam), a sensing layer comprising the matrix of sensors 202, and a substantially stiff underlayment 222 (e.g., a plastic such as ABS, rubber, or foam). The sensors 202 are arranged in a grid (such as 4×4 equally spaced pattern) to enables the sensors 202 to individually sense pressure applied to different physical locations. In an embodiment, the size of the sensing device 150 is substantially matched to a size of the applicable sitting area of the wheelchair user. Other architectural components of the sensing device 150 such as the battery 204, the communication interface 206, the controller 208, the CPU 216, and the supporting circuitry 218 may be integrated on one or more printed circuit boards (PCBs) on the sensing layer or elsewhere in the device 202.

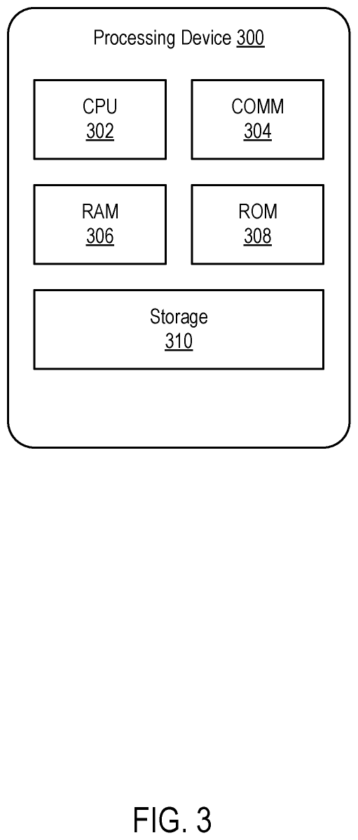

FIG. 3 is a block diagram illustrating an example embodiment of a processing device 300 that could be utilized as the user device 130, the caretaker device 140, or the compliance server 110. The processing device 300 may include conventional computing components such as a central processing unit (CPU) 302, random access memory (RAM) 306, read only memory (ROM) 308, storage 310 (e.g., a disk drive or solid state device), and a communication interface 304 for communicating with the network 120 and/or the sensing device 150. The processing device 300 may implement the compliance management application 132, caretaker application 142, or server application 112 as a set of instructions stored to storage 310 (e.g., a non-transitory computer readable storage medium), loaded in RAM 306, and executed by the CPU 302 to carry out the functions attributed to the applications described herein.

FIG. 4 is a block diagram illustrating an example embodiment of a compliance management application 400. The compliance management application 400 may wholly or partially execute on the user device 130. However, in other embodiments, various modules of the compliance management application 400 may instead be executed on the sensing device 150, the compliance server 110, the caretaker device 140, or another device.

The compliance management application 400 includes a user interface module 402, a connection management module 404, a sensor data processing module 406, a compliance assessment module 408, and a notification module 410. In alternative embodiments, the compliance management application 400 may include different or additional modules.

The user interface module 402 provides display elements and controls to enable a user to interact with the compliance management system 100. For example, the user interface module 402 may enable a user to log into the application 400, access a user profile, and provide various patient-specific information such as name, height, weight, pressure ulcer history, incontinence history, user and caretake notification preferences, and/or other demographic or medical information helpful to developing shift recommendations. The user interface 402 may furthermore present notifications to the user (e.g., visual, audible, and/or haptic) based on the shift recommendations and may present various analytical information relating to compliance history.

The connection management module 404 manages connections between devices such as between the user device 130 and the sensing device 150 and/or between the user device 130 and the network 120. In an example embodiment, the connection management module 404 may control pairing of the user device 130 with the sensing device 150 via Bluetooth or another wireless connection. Alternatively, the connection management module 404 may manage a wired connection between the user device 130 and the sensing device 150. The connection module 404 may furthermore control timing of data transfers between the sensing device 150 and the user device 130 and between the user device 130 and the compliance server 110 as described further below.

The sensor data processing module 406 processes the sensing data received from the sensing device 150. In an embodiment, the sensor data processing module 406 receives the raw sensor data from the sensing device 150 and generates various behavior parameters. For example, the sensor data processing module 406 may identify when pressure is present or absent in various areas (or the relative pressures in different areas) and develop pressure distribution profiles for different users. The sensor data processing module 406 may furthermore identify time periods when the user is seated on the device 150 and time periods when the user is not seated. Here, the sensor data processing module 406 may detect that the user is seated when at least a predefined number of sensors 202 (e.g., all of the sensors, 90% of the sensors, half of the sensors, a quarter of the sensors, a single sensor, etc.) detect a pressure level that meets at least a predefined pressure threshold indicative of the individual sitting on the device 150. The sensor data processing module 406 may also detect when the user is performing weight shift exercises and specific shift data characterizing those movements. Here, the sensor data processing module 406 may detect time periods when weight shifts from one part of the sensor array 202 to another part of the sensor array 202. For example, the sensor data processing module 406 detects when at least a predefined pressure change occurs in at least a predefined number of sensors 202 and is maintained over at least a predefined time threshold.

The compliance assessment module 408 obtains a set of shift recommendations for the user and compares the detected weight shifts performed by the user to the shift recommendations to generate one or more compliance metrics. The set of shift recommendations may comprise, for example, a user-specific set of recommendations provided by the user, the caretaker, or another individual. Alternatively, in the absence of a user-specific set of recommendations, a default set of recommendations may be applied based on generally accepted medical practices. In some embodiments, the default set of recommendations may be partially tailored to the patient based on biometric and/or demographic information in the user's profile. For example, the shift recommendations may be based on user-specified areas of discomfort, pressure ulcer history, or other data. Furthermore, the shift recommendations may be based in part, on the observed compliance of the individual with past recommendations. For example, the recommended frequency and/or duration of shift exercises may automatically decrease in response to observing a low compliance level. The shift recommendations may furthermore be based in part on individual user preferences configured via the user interface module 402.

The shift recommendations may be specified by at least one of a recommendation minimum duration of weight shifts to be performed during the exercises, a frequency or non-periodic schedule for performing the weight shift exercises, minimum change in weight distribution for each weight shift exercise, or other criteria. An example weight shift recommendation may be for the patient to perform a 10 second weight shift every 30 minutes. In other embodiments, more specific recommendations may be provided such as, shift weight from the left side to the right side.

The compliance assessment module 408 may generate various metrics indicative of compliance level. In one embodiment, the compliance assessment module 308 generates a binary value associated with each recommended exercise indicative of whether or not the criteria for that exercise was met. The binary values may be aggregated over one or more time windows to indicate a compliance level for that time window. For example, if a user met the criteria for 50% of the recommended weight shift exercises over a 24 hour period, a compliance score of 50% may be computed for that period. Alternatively, compliance scores may be computed over more or less granular time periods (e.g., each hour, 4 hour period, 24 hour period, week, month, or year).

In other embodiments, the compliance assessment module 408 may generate compliance values for each recommended exercise as a non-binary exercise score on a continuous or discrete scoring scale. Here, the exercise score may assess a level of compliance with the set of criteria for the exercise even if all of the criteria is not met. For example, if a weight shift recommendation specifies a duration of 10 seconds and the user performs the exercise for only 8 seconds, a score of 80% (or alternatively 8 out 10 or 80 out of 100) may be assigned for that exercise. Alternatively, scoring may be based on other criteria such as how closely the change in weight distribution matches a desired profile, or based on a combination of criteria. The scores may similarly be aggregated over long time periods (e.g., by summing or averaging the scores) to generate compliance scores for different time periods.

In an embodiment, the compliance assessment module 408 may dynamically update compliance recommendations based on compliance level or other information. Updated recommendations may be generated locally or may be communicated by the compliance server 110 as described above. In one embodiment, compliance recommendations may be specific to a particular pressure distribution profile observed for a particular user. For example, if the user is observed to consistently have certain areas of high pressure, the recommendations may suggest weight exercises that relieve those specific areas.

The notification module 410 generates notifications associated with managing weight shift compliance. The notifications may include, for example, reminders when it is time to perform weight shift exercises based on the recommendations and feedback related to the user's compliance scores. Compliance feedback may be provided substantially in real-time after each scheduled exercise and/or periodically to indicate compliance over longer time periods. The types of frequencies of notifications may be customized by the user and/or the caretaker based on configured preferences.

In further embodiments, the notification module 410 may present real-time sensor data. For example, the notification module 410 may present a user interface showing a grid representative of the pressure sensor matrix in which each element in the grid indicates the observed sensor readings. Here, the pressure levels may be represented, for example, using color-coded squares, numerical values, or other indicators. Alternatively, instead of presenting real-time data, the grid may represent an aggregation of historical data (e.g., average pressure over the last 24 hours, last week, last month, last year, etc.).

In yet further embodiments, the notification module 410 may present analytical data such as indications of which areas are subject to the highest pressure on a daily basis, the user's daily or weekly average weight shift compliance level, the user's daily average time seated in the wheelchair, etc.

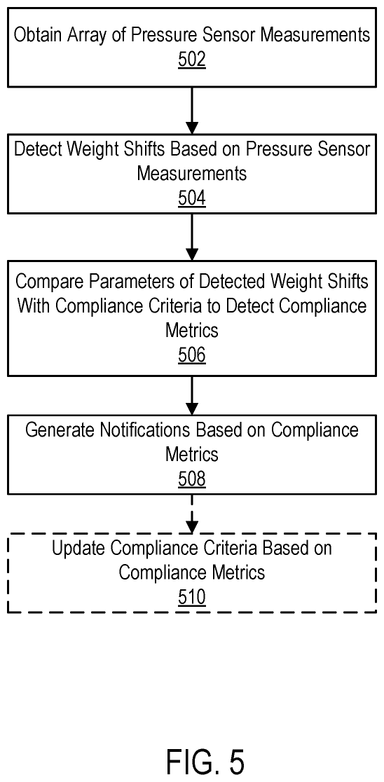

FIG. 5 illustrates an example embodiment of a process for managing shift compliance. A compliance management application 400 receives 502 measurements from an array of pressure sensors in a sensing device. The compliance management application 400 detects 504 weight shifts based on the sensor data (e.g., characterized by frequency, duration, and/or change in pressure distribution profiles). The compliance management application 400 compares 506 parameters of the detected weight shifts with compliance criteria to detect the compliance metrics as described above. The compliance management application 400 then generates notifications 508 indicative of the compliance metrics. Optionally, compliance criteria may be updated 510 based on instructions from the compliance server 110 received in response to the compliance metrics.

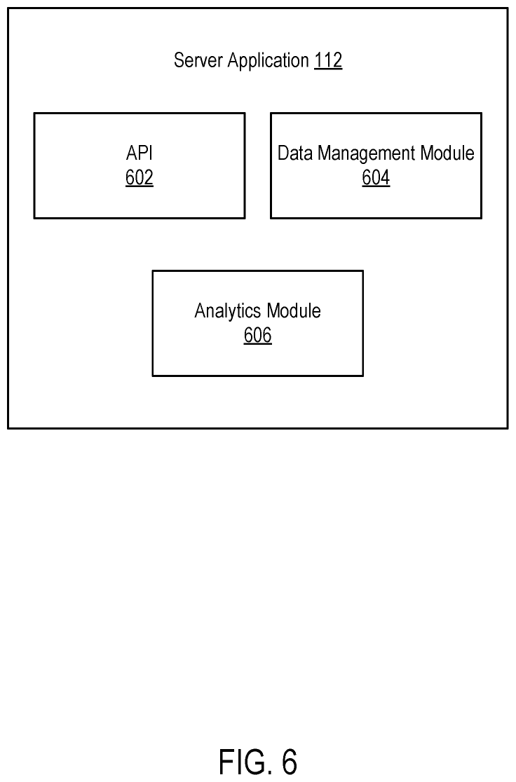

FIG. 6 illustrates an example embodiment of a server application 112. The server application 112 includes an application programming interface (API) 602, a data management module 604, and an analytics module 606. The API 602 receives the processed sensor data from the user device 130 (or directly from the sensing device 150), formats the data, and provide the data to the data management module 604 for secure storage (e.g., in a cloud storage database). The analytics module 606 may periodically access the sensor data via the data management module 604 and may generate various analytical insights based on the sensor data. The analytical insight data may be further communicated via the API 602 to the user device 130 and/or the caretaker device 140. The compliance management application 132 and caretaker application 142 may request different types of analytical insight data from the server application 112 and may display and/or otherwise utilize the data differently.

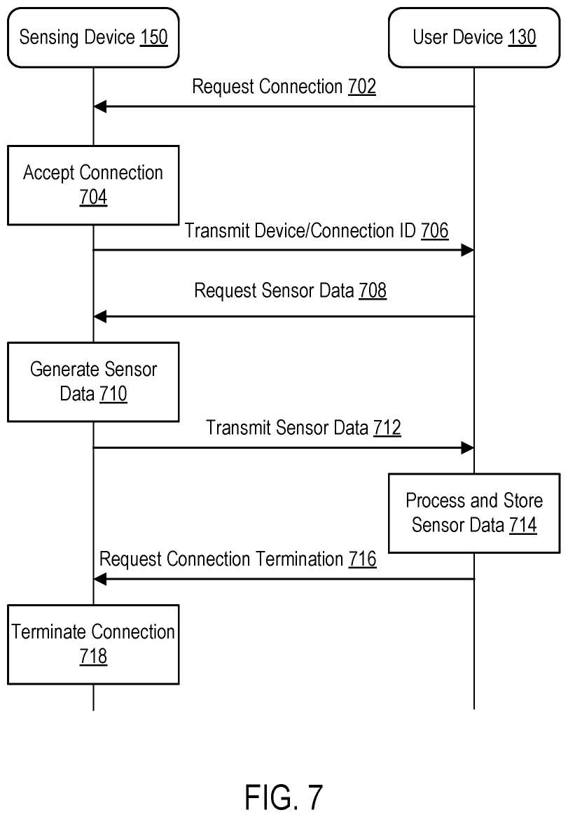

FIG. 7 is a messaging diagram illustrating a protocol for establishing a connection between the sensing device 150 and the user device 130. The user device 130 requests 702 a connection to the sensing device 150. The sensing device 150 validates the connection request 302, and if valid, accepts 704 the request. The sensing device 150 then transmits 706 a unique connection identifier to the user device 130 to complete the connection. Once connected, the user device 130 can issue various commands to the sensing device 150 to obtain sensed data. For example, the user device 130 may issue a sensor data request command 708 that causes the sensing device 150 to generate 710 the sensor data (e.g., by reading directly from the sensors 202 or data from the data cache 214) and transmit 712 the sensed data to the user device 130 where it is processed and stored 714. When the connection is no longer desired (e.g., because sufficient data has been read), the user device 130 may issue a connection termination command 716 that causes the connection to be terminated 718.

In various alternative embodiments, data may be obtained from the sensing device 150 according to a different protocol. For example, in one embodiment, the sensing device 150 may automatically push data to the user device 130 without necessarily receiving request commands 708. Furthermore, in different embodiments, the connection request 702 and/or connection termination command 716 may instead be initiated by the sensing device 150.

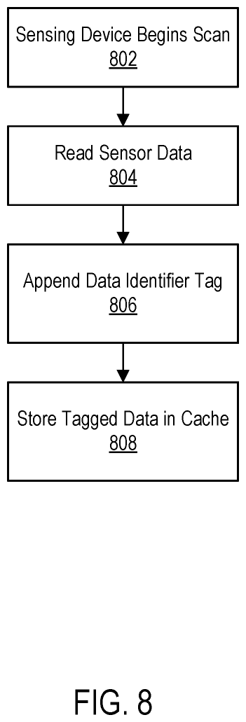

FIG. 8 is a flowchart illustrating an example embodiment of a process performed by the sensing device 150 to collect sensed data for transmission to the user device 130. The sensing device 150 initiates 802 a scan and reads 804 sensed pressure data from each of the pressure sensors 202. The pressure sensors 202 may be read substantially in parallel (i.e., within a very brief time window (e.g., less than 1 second) of each other) or may be sequentially scanned at different intervals. The sensing device 150 appends 806 one or more data identifier tags to each sensor reading. The tags may include, for example, a timestamp (obtained from a timer of the supporting circuitry 218) or other monotonic tag that distinguishes relative timing of sensor readings. The tags may also identifier which sensor or region of the sensing device 150 each data point is captured from. The tagged data is stored 808 in the cache 214 or may be directly sent to the user device 150.

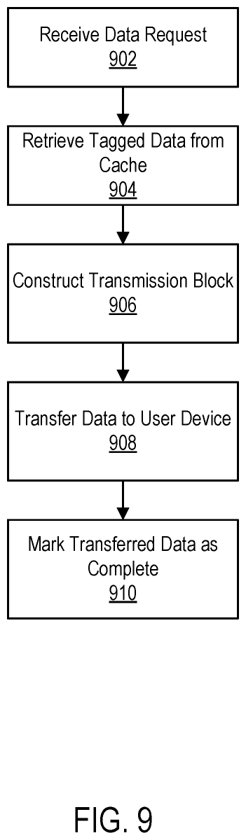

FIG. 9 is a flowchart illustrating an example embodiment of a process performed by the sensing device 150 for transmitting cached sensor data to the user device 130. The sensing device 150 receives 902 a request from the user device 130 for the sensed data. The sensing device 150 retrieves 904 the tagged data from the cache 214, constructs 906 a transmission block, and transfers 908 the data to the user device 130. The transmission block may include the sensed data and various metadata such as the identifier tags, transmission size information, remaining transferable data, or other metadata. Upon completion, the sensing device 150 marks 910 the data as complete and may enable the data to be overwritten in the cache 214.

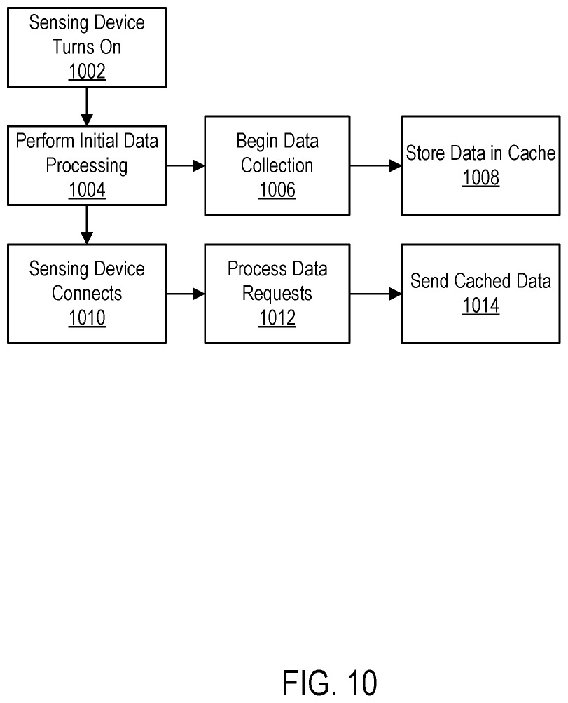

FIG. 10 is a flowchart illustrating a further embodiment of a process performed by the sensing device 150. In this process, the sensing device 150 initially turns on 1002 (from a sleep or off state) and begins initial processing 1004 of data. This may include, for example, marking stale data (i.e., data that has already been transferred or is older than a predefined age) as invalid, initializing pointers, and initializing the timing mechanism. The sensing device 150 then begins 1006 periodic data collection and storing 1008 collected data to the data cache 214. In parallel, the sensing device 150 may connect 1010 to the user device to begin processing 1012 received data transfer requests from the user device 130, and sending 1014 the cached data to the user device 130.

Embodiments of the described shift compliance management system 100 and corresponding processes may be implemented by one or more computing systems. The one or more computing systems include at least one processor and a non-transitory computer-readable storage medium storing instructions executable by the at least one processor for carrying out the processes and functions described herein. The computing system may include distributed network-based computing systems in which functions described herein are not necessarily executed on a single physical device. For example, some implementations may utilize cloud processing and storage technologies, virtual machines, or other technologies.

The foregoing description of the embodiments has been presented for the purpose of illustration; it is not intended to be exhaustive or to limit the embodiments to the precise forms disclosed. Persons skilled in the relevant art can appreciate that many modifications and variations are possible in light of the above disclosure.

Some portions of this description describe the embodiments in terms of algorithms and symbolic representations of operations on information. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a tangible non-transitory computer readable storage medium or any type of media suitable for storing electronic instructions, and coupled to a computer system bus. Furthermore, any computing systems referred to in the specification may include a single processor or may be architectures employing multiple processor designs for increased computing capability.

Finally, the language used in the specification has been principally selected for readability and instructional purposes, and it may not have been selected to delineate or circumscribe the inventive subject matter. It is therefore intended that the scope is not limited by this detailed description, but rather by any claims that issue on an application based hereon. Accordingly, the disclosure of the embodiments is intended to be illustrative, but not limiting, of the scope of the invention, which is set forth in the following claims.

1. A method for managing shift compliance for preventing pressure ulcers in a wheelchair user, the method comprising:
receiving by a compliance management application at each of a sequence of time points, a two-dimensional array of pressure measurements from a sensor grid placed underneath a gluteal region;
processing the pressure measurements to identify weight shifts meeting detection criteria;
comparing the weight shifts with compliance criteria to detect a compliance level of the wheelchair user; and
generating electronic notifications based on the detected compliance level.

2. The method of claim 1, wherein receiving the pressure measurements comprises:
receiving, for each pressure sensor in the sensor grid, a time-averaged pressure measurement averaged over time by a microcontroller integrated in the sensor grid.

3. The method of claim 1, wherein receiving the pressure measurements comprises:
receiving the pressure measurements over a Bluetooth Low Energy connection with the sensor grid.

4. The method of claim 1, wherein receiving the pressure measurements comprises:
receiving the pressure measurements at a user device in substantially real-time while the user device is communicatively coupled with the sensor grid.

5. The method of claim 1, wherein receiving the pressure measurements comprises:
receiving the pressure measurements at a user device in batch upon the user device establishing a connection with the sensor grid.

6. The method of claim 1, wherein processing the pressure measurements comprises:
detecting at least a first time period when the wheelchair user is seated and detecting at least a second time period when the wheelchair user is not seated.

7. The method of claim 1, wherein processing the pressure measurements comprises:
detecting a shift period when the patient's weight shifts from a baseline seated position.

8. The method of claim 1, wherein processing the pressure measurements comprises:
detecting a pressure change of at least a predefined magnitude relative to a baseline sitting pressure that occurs in at least a predefined number of pressure sensors and lasts at least a predefined time period.

9. The method of claim 1, wherein comparing the weight shifts with compliance criteria comprises:
obtaining a predefined recommended duration for a first time interval; and
detecting, during the first time interval, a duration of the weight shift; and
determining the compliance level based on the duration of the weight shift relative to the predefined recommended duration.

10. The method of claim 1, wherein comparing the weight shifts with the compliance criteria comprises:
determining respective compliance levels for each of a sequence of time intervals.

11. The method of claim 1, wherein generating the electronic notifications comprises:
communicating the compliance levels to a remote processing server; and
obtaining information for the electronic notifications from the remote processing server.

12. The method of claim 1, where generating the electronic notifications comprises:
applying a recommendation algorithm to generate, based on the two-dimensional array of pressure measurements, a patient-specific shift recommendation; and
sending the patient-specific shift recommendation to a user device.

13. A non-transitory computer-readable storage medium storing instructions for managing shift compliance for preventing pressure ulcers in a wheelchair user, the instructions when executed by a processor causing the processor to perform steps including:
receiving by a compliance management application at each of a sequence of time points, a two-dimensional array of pressure measurements from a sensor grid placed underneath a gluteal region;
processing the pressure measurements to identify weight shifts meeting detection criteria;
comparing the weight shifts with compliance criteria to detect a compliance level of the wheelchair user; and
generating electronic notifications based on the detected compliance level.

14. The non-transitory computer-readable storage medium of claim 13, wherein processing the pressure measurements comprises:
detecting at least a first time period when the wheelchair user is seated and detecting at least a second time period when the wheelchair user is not seated.

15. The non-transitory computer-readable storage medium of claim 13, wherein processing the pressure measurements comprises:
detecting a shift period when the patient's weight shifts from a baseline seated position.

16. The non-transitory computer-readable storage medium of claim 13, wherein processing the pressure measurements comprises:
detecting a pressure change of at least a predefined magnitude relative to a baseline sitting pressure that occurs in at least a predefined number of pressure sensors and lasts at least a predefined time period.

17. The non-transitory computer-readable storage medium of claim 13, wherein comparing the weight shifts with compliance criteria comprises:
obtaining a predefined recommended duration for a first time interval; and
detecting, during the first time interval, a duration of the weight shift; and
determining the compliance level based on the duration of the weight shift relative to the predefined recommended duration.

18. The non-transitory computer-readable storage medium of claim 13, wherein comparing the weight shifts with the compliance criteria comprises:
determining respective compliance levels for each of a sequence of time intervals.

19. A shift compliance management system comprising:
a sensing device including:
a lower layer comprising a substantially rigid material;
an interior sensor layer comprising a sensor grid for individually sensing pressure at different locations; and
an upper layer comprising a cushioning material; and
a processing device comprising:
a processor; and
a non-transitory computer-readable storage medium storing instructions for managing shift compliance for preventing pressure ulcers in a wheelchair user, the instructions when executed by a processor causing the processor to perform steps including:
receiving by a compliance management application at each of a sequence of time points, a two-dimensional array of pressure measurements from the sensor;
processing the pressure measurements to identify weight shifts meeting detection criteria;
comparing the weight shifts with compliance criteria to detect a compliance level of the wheelchair user; and
generating electronic notifications based on the detected compliance level.

20. The shift compliance management system of claim 19, wherein the sensing device further comprises:
a controller including a data cache for temporarily storing the pressure measurements obtained from the sensors; and
a communication interface for wireless communicating the sensor measurements from the data cache to the processing device.
